(19) United States
(12) Patent Application Publication
(10) Pub. No.: US 2004/0047896 A1
(43) Pub. Date: Mar. 11, 2004

(54) Composition for improving age-related physiological deficits and increasing longevity

(76) Inventors: Armand Malnoe, Epalinges (CH); Sylvie Pridmore-Merten, Lausanne (CH)

Correspondence Address: WINSTON & STRAWN, PATENT DEPARTMENT, 1400 L STREET, N.W., WASHINGTON, DC, 20005-3502, US

(21) Appl. No.: 10/656,955

(22) Filed: Sep. 5, 2003

Related U.S. Application Data
(63) Continuation of application No. PCT/EP02/02862, filed on Mar. 7, 2002.

(30) Foreign Application Priority Data
Mar. 9, 2001 (EP) 01200871.0

(51) Int. Cl. A61K 38/05; A61K 31/7076; A61K 31/202; A61K 31/198

(52) U.S. Cl. 424/439; 514/565; 514/560; 514/562; 514/46; 514/440; 514/18

(57) ABSTRACT

The invention relates to a food composition intended to prevent or restore age-related functional deficits in mammals, which comprises a combination being able to mimic the effects of caloric restriction on gene expression. The combination contains at least one molecule that stimulates energy metabolism of the cell and at least one antioxidant. Also, methods of treating age-related functional deficits in mammals by administering the combination or a food composition containing the same.

### CROSS-REFERENCE TO RELATED APPLICATION

The present application is a continuation of International application PCT/EP02/02862 filed Mar. 7, 2002, the entire content of which is expressly incorporated herein by reference thereto.

### TECHNICAL FIELD

This invention relates to a composition for improving age-related physiological deficits and extending life span in mammals. The invention also relates to a method for improving the condition of elderly mammals, particularly by preventing or restoring the age-related metabolic changes particularly those bound to mitochondria dysfunction.

### BACKGROUND OF THE INVENTION

Elderly mammals often become frail in their last few years of life. From an appearance point of view, they become thin and have poor skin and coat condition. Other symptoms include joint stiffness, loss of lean body mass, energy loss, weight gain, neurological disorders and digestive system problems.

Certain of these problems may be effectively treated using medication but a better alternative would be to delay the onset of these problems, or treat these problems, through the diet. In particular, elderly animals should be fed a balanced, maintenance food that contains high quality protein, lower amounts of fat to reduce energy intake, dietary fiber, and antioxidants. However, despite the use of balanced, maintenance foods, the condition of elderly animals may deteriorate rapidly.

On the molecular level, it is known that mitochondria function is impaired during aging and this is associated with important functional deficits (both physical and cognitive) and the development of degenerative diseases.

Indeed, mitochondria generate most of the energy of the cell primarily through oxidative phosphorylation, a complex process that uses electrons generated through oxidation of glucose and fatty acids to generate ATP. Proteins of the mitochondria oxidative phosphorylation complex have been shown to be impaired upon aging, leading to a higher production of reactive oxygen species (ROS) and a decrease in efficiency of energy production. Free radical produced by aerobic respiration cause cumulative oxidative damages resulting in aging and cell death. The biggest impact of age-related increase in ROS will be on somatic tissues composed of post-mitotic non-replicative cells (muscles: cardiac and skeletal, nervous tissues: brain, retinal pigment epithelium).

Numerous age-related changes have been reported in mitochondria. Oxidative damage to mitochondria DNA (mt DNA) increases with aging (Beckman K B, Ames B N (1999) Mutat Res. 424 (1-2):51-8) along with the oxidation of glutathione (GSH) a major intracellular antioxidant system, which plays an important role in protection against age-related mt DNA oxidative damage. A substantial increase in protein oxidation is also observed upon aging (Stadtman E R. (1992), Science 257 (5074):1220-4). Age-related increase in the amount of long chain polyunsaturated fatty acids has been linked to the high peroxidizability of the mitochondria lipids upon aging. This is well illustrated by the change in the composition of cardiolipin, a phospholipid found principally in mitochondria, which fatty acid composition tends to shift towards a more unsaturated state with substitution of 18:2 acyl chains with the more peroxidizable 22:4 and 22:5 upon aging (Laganiere S, Yu B P (1993), Gerontology 39 (1):7-18). The mitochondria content in cardiolipin has also been reported to decrease with age. Cardiolipin interacts with many components of the mitochondria inner membrane such as Cytochrome oxidase, transporters/translocators (ADP/ATP, phosphate, pyruvate, camitine, etc) and plays an active role in their activity (Hoch F L. (1992) Biochim Biophys Acta. 1113 (1):71-133; Paradies G, Ruggiero F M. (1990) Biochim Biophys Acta. 1016(2):207-12). The mitochondria energy metabolism depends upon the transport of metabolites such as pyruvate across the mitochondria inner membrane. Pyruvate transport is carrier-mediated (Hoch F L. (1988) Prog Lipid Res. 27 (3):199-270) and a requirement for cardiolipin has been demonstrated for optimal pyruvate translocase activity (Paradies G, Ruggiero F M. (1990) Biochim Biophys Acta. 1016 (2):207-12). Other modifications such as decrease in mitochondria membrane potential and morphological changes (swelling, altered cristae, matrix vacuolisation) are associated with chronic oxidative stress and aging.

Several dietary interventions have been described that restore the age-related metabolic changes and increase longevity.

For example, long-term caloric restriction (CR) initiated before mid-life, retards aging and has multiple effects on the metabolism of the cell. Indeed, CR decreases oxidative damage to DNA, proteins and lipids in rodents (Shigenaga M K, Ames B N. (1994) in: Natural Antioxidants in Human Health and Disease, B. Frei editor, Academic Press, New York. pp 63-106) increases motor activity in rodents, reduces fiber loss and the age-related accumulation of dysfunctional fibers (Aspnes L E et al. (1997) FASEB J. 11 (7):573-81). However life long food restriction in pets is both unpractical and not well perceived by pet owners.

Therefore there is a need for non-restricted and efficient nutritional ways of improving age-related physiological deficits and extending life span in humans and animals, more particularly pets.

### SUMMARY OF THE INVENTION

Accordingly, in a first aspect, the present invention provides a food composition intended to prevent or restore age-related functional deficits in mammals by reversing age-related gene expression alterations. The food composition comprises an edible substance and a combination that is able to mimic the effects of caloric restriction on gene expression. The combination contains at least one molecule that stimulates energy metabolism of the cell and at least one antioxidant.

Indeed, it has been surprisingly found that the effects of caloric restriction on gene expression can be mimicked by nutritional interventions that do not limit calorie intake but result in improved mitochondria function. In fact, it is possible to target mitochondria function through dietary intervention and have an impact on genes linked to energy metabolism and longevity.

In a preferred embodiment, the molecule that stimulates energy metabolism is any nutrient improving energy production in mitochondria, such as L-camitine, creatine, fatty acids (mono and polyunsaturated, particularly omega-3 fatty acids), cardiolipin, nicotinamide, carbohydrate and natural sources thereof, for example.

The antioxidant aims to prevent or at least reduce oxidative damage that can result from the disruption of the ATP/ADP and/or NAD+/NADH homeostasis due to the increased substrate availability/utilization in the aged mitochondria. Among antioxidants: sources of thiols, compounds that decrease protein oxidation and compounds that upregulate cell antioxidant defenses are preferably used.

The food composition may be a complete and nutritionally balanced food for human or animal. It can also be a dietary supplement, for example.

The food composition according to the present invention can prevent or delay mitochondrial dysfunctions occuring during aging by modulating and/or regulating expression of genes linked to energy metabolism. It can also provide multiple benefits by improving age-related functional deficits e.g. in skeletal and cardiac muscle function, vascular function, cognitive function, vision, hearing, olfaction, skin and coat quality, bone and joint health, renal health, gut function, immune function, insulin sensitivity, inflammatory processes, cancer incidence and ultimately increasing longevity in pets.

In another aspect, this invention relates to the use of a combination that is able to mimic the effects of caloric restriction on gene expression, which comprises at least one molecule that stimulates energy metabolism of the cell and at least one antioxidant, for the preparation of a composition intended to prevent or restore age-related functional deficits in mammals.

In a further aspect, this invention provides a method to prevent or restore age-related functional deficits in mammals, comprising administering to the mammal, a food composition comprising a combination being able to mimic the effects of caloric restriction on gene expression. Again, this combination contains at least one molecule that stimulates energy metabolism of the cell and at least one antioxidant.

The composition may be administered to the mammal as a supplement to the normal diet or as a component of a nutritionally complete food. It is preferred to include the nutritional agent in a nutritionally complete food.

Administering to a mammal, a food composition as described above, results in an improved mitochondria function, also mimicking the effects of caloric restriction on gene expression without limiting calorie intake.

### DETAILED DESCRIPTION OF THE PREFERRED EMBODIMENTS

With respect to the first object of the present invention, a food composition intended to prevent or restore age-related functional deficits in mammals by reversing age-related gene expression alterations, which comprises a combination being able to mimic the effects of caloric restriction on gene expression, said combination containing at least one molecule that stimulates energy metabolism of the cell and at least one antioxidant.

In a preferred embodiment, the molecule stimulates in particular energy metabolism of the mitochondria.

Indeed, it has been surprisingly found that the effects of caloric restriction on gene expression can be mimicked by nutritional interventions that do not limit calorie intake but result in improved mitochondria function.

The molecule that stimulates energy metabolism of the cell and in particular the energy metabolism of the mitochondria may be L-carnitine, creatine, fatty acids (mono or polyunsaturated fatty acids, particularly omega-3 fatty acids), cardiolipin, nicotinamide, carbohydrate and natural sources thereof, for example.

Preferably, the amount of said molecule is of at least 1 mg per kg of body weight per day, more preferably from 1 mg to 1 g per kg of body weight per day.

The antioxidants are compounds that decrease protein oxidation (e.g. prevent formation of protein carbonyls). They may be sources of thiols (e.g. Lipoic acid, cysteine, cystine, methionine, S-adenosyl-methionine, taurine, glutathione and natural sources thereof), or compounds that upregulate their biosynthesis in vivo, for example.

The antioxidant according to the invention may be used either alone or in association with other antioxidants such as vitamin C, vitamin E (tocopherols and tocotrienols), carotenoids (carotenes, lycopene, lutein, zeaxanthine.) ubiquinones (e.g. CoQ10), tea catechins (e.g. epigallocatechin gallate), coffee extracts containing polyphenols and/or diterpenes (e.g. kawheol and cafestol), ginkgo biloba extracts, grape or grape seed extracts rich in proanthocyanidins, spice extracts (e.g. rosemary), soy extracts containing isoflavones and related phytoestrogens and other sources of flavonoids with antioxidant activity, compounds that upregulate cell antioxidant defense (e.g. ursodeoxycholic acid for increased glutathione S-transferase, ursolic acid for increased catalase, ginseng and gingenosides for increase superoxide dismutase and natural sources thereof i.e. herbal medicines).

Preferably, the amount of the antioxidant is of at least 0.025 mg per kg of body weight per day, more preferably from 0.025 mg to 250 mg per kg of body weight per day.

The food composition may be a complete and nutritionally balanced food. It can also be a dietary supplement, for example.

In one embodiment, a nutritionally complete pet food can be prepared. The nutritionally complete pet food may be in any suitable form; for example in dried form, semi-moist form or wet form; it may be a chilled or shelf stable pet food product. These pet foods may be produced as is conventional. Apart from the combination according to the invention, these pet foods may include any one or more of a carbohydrate source, a protein source and lipid source.

Any suitable carbohydrate source may be used. Preferably the carbohydrate source is provided in the form of grains, flours and starches. For example, the carbohydrate source may be rice, barley, sorghum, millet, oat, corn meal or wheat flour. Simple sugars such as sucrose, glucose and corn syrups may also be used. The amount of carbohydrate provided by the carbohydrate source may be selected as desired. For example, the pet food may contain up to about 60% by weight of carbohydrate.

Suitable protein sources may be selected from any suitable animal or vegetable protein source; for example muscular or skeletal meat, meat and bone meal, poultry meal, fish meal, milk proteins, corn gluten, wheat gluten, soy flour, soy protein concentrates, soy protein isolates, egg proteins, whey, casein, gluten, and the like. For elderly animals, it is preferred for the protein source to contain a high quality animal protein. The amount of protein provided by the protein source may be selected as desired. For example, the pet food may contain about 12% to about 70% by weight of protein on a dry basis.

The pet food may contain a fat source. Any suitable fat source may be used both animal fats and vegetable fats. Preferably the fat source is an animal fat source such as tallow. Vegetable oils such as corn oil, sunflower oil, safflower oil, rape seed oil, soy bean oil, olive oil and other oils rich in monounsaturated and polyunsaturated fatty acids, may also be used. In addition to essential fatty acids (linoleic and alpha-linoleic acid) the fat source may include long chain fatty acids. Suitable long chain fatty acids include, gamma linoleic acid, stearidonic acid, arachidonic acid, eicosapentanoic acid, and docosahexanoic acid. Fish oils are a suitable source of eicosapentanoic acids and docosahexanoic acid. Borage oil, blackcurrent seed oil and evening primrose oil are suitable sources of gamma linoleic acid. Rapeseed oil, soybean oil, linseed oil and walnut oil are suitable sources of alpha-linoleic acid. Safflower oils, sunflower oils, corn oils and soybean oils are suitable sources of linoleic acid. Olive oil, rapeseed oil (canola) high oleic sunflower and safflower, peanut oil, rice bran oil are suitable sources of monounsaturated fatty acids. The amount of fat provided by the fat source may be selected as desired. For example, the pet food may contain about 5% to about 40% by weight of fat on a dry basis. Preferably, the pet food has a relatively reduced amount of fat.

The pet food may contain other active agents such as long chain fatty acids. Suitable long chain fatty acids include alpha-linoleic acid, gamma linoleic acid, linoleic acid, eicosapentanoic acid, and docosahexanoic acid. Fish oils are a suitable source of eicosapentanoic acids and docosahexanoic acid. Borage oil, blackcurrent seed oil and evening primrose oil are suitable sources of gamma linoleic acid. Safflower oils, sunflower oils, corn oils and soybean oils are suitable sources of linoleic acid.

The choice of the carbohydrate, protein and lipid sources is not critical and will be selected based upon nutritional needs of the animal, palatability considerations, and the type of product produced. Further, various other ingredients, for example, sugar, salt, spices, seasonings, vitamins, minerals, flavoring agents, gums, prebiotics and probiotic micro-organisms may also be incorporated into the pet food as desired The prebiotics may be provided in any suitable form. For example, the prebiotic may be provided in the form of plant material, which contains the prebiotic. Suitable plant materials include asparagus, artichokes, onions, wheat, yacon or chicory, or residues of these plant materials. Alternatively, the prebiotic may be provided as an inulin extract or its hydrolysis products commonly known as fructooligosaccharides, galacto-oligosaccarides, xylo-oligosaccharides or oligo derivatives of starch. Extracts from chicory are particularly suitable. The maximum level of prebiotic in the pet food is preferably about 20% by weight; especially about 10% by weight. For example, the prebiotic may comprise about 0.1% to about 5% by weight of the pet food. For pet foods which use chicory as the prebiotic, the chicory may be included to comprise about 0.5% to about 10% by weight of the feed mixture; more preferably about 1% to about 5% by weight.

The probiotic microorganism may be selected from one or more microorganisms suitable for animal consumption and which is able to improve the microbial balance in the intestine. Examples of suitable probiotic micro-organisms include yeast such as Saccharomyces, Debaromyces, Candida, Pichia and Torulopsis, moulds such as Aspergillus, Rhizopus, Mucor, and Penicillium and Torulopsis and bacteria such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus and Lactobacillus. Specific examples of suitable probiotic microorganisms are: Saccharomyces cereviseae, Bacillus coagulans, Bacillus licheniformis, Bacillus subtilis, Bifidobacterium bifidum, Bifidobacterium infantis, Bifidobacterium longum, Enterococcus faecium, Enterococcus faecalis, Lactobacillus acidophilus, Lactobacillus alimentarius, Lactobacillus casei subsp. casei, Lactobacillus casei Shirota, Lactobacillus curvatus, Lactobacillus delbruckii subsp. lactis, Lactobacillus farciminus, Lactobacillus gasseri, Lactobacillus helveticus, Lactobacillus johnsonii, Lactobacillus reuteri, Lactobacillus rhamnosus (Lactobacillus GG), Lactobacillus sake, Lactococcus lactis, Micrococcus varians, Pediococcus acidilactici, Pediococcus pentosaceus, Pediococcus acidilactici, Pediococcus halophilus, Streptococcus faecalis, Streptococcus thermophilus, Staphylococcus carnosus, and Staphylococcus xylosus. The probiotic microorganisms may be in powdered, dried form; especially in spore form for micro-organisms which form spores. Further, if desired, the probiotic micro-organism may be encapsulated to further increase the probability of survival; for example in a sugar matrix, fat matrix or polysaccharide matrix. If a probiotic micro-organism is used, the pet food preferably contains about 10⁴ to about 10¹⁰ cells of the probiotic micro-organism per gram of the pet food; more preferably about 10⁶ to about 10⁸ cells of the probiotic micro-organism per gram. The pet food may contain about 0.5% to about 20% by weight of the mixture of the probiotic micro-organism; preferably about 1% to about 6% by weight; for example about 3% to about 6% by weight.

For elderly pets, the pet food preferably contains proportionally less fat than pet foods for younger pets. Further, the starch sources may include one or more of oat, rice, barley, wheat and corn.

For dried pet foods a suitable process is extrusion cooking, although baking and other suitable processes may be used. When extrusion cooked, the dried pet food is usually provided in the form of a kibble. If a prebiotic is used, the prebiotic may be admixed with the other ingredients of the dried pet food prior to processing. A suitable process is described in European patent application No 0850569. If a probiotic micro-organism is used, the organism is best coated onto or filled into the dried pet food. A suitable process is described in European patent application No 0862863.

For wet foods, the processes described in U.S. Pat. Nos. 4,781,939 and 5,132,137 may be used to produce simulated meat products. Other procedures for producing chunk type products may also be used; for example cooking in a steam oven. Alternatively, loaf type products may be produced by emulsifying a suitable meat material to produce a meat emulsion, adding a suitable gelling agent, and heating the meat emulsion prior to filling into cans or other containers.

In another embodiment, a food composition for human consumption is prepared. This composition may be a nutritional complete formula, a dairy product, a chilled or shelf stable beverage, soup, a dietary supplement, a meal replacement, and a nutritional bar or a confectionery.

Apart from the combination according to the invention, the nutritional formula may comprise a source of protein. Dietary proteins are preferably used as a source of protein. The dietary proteins may be any suitable dietary protein; for example animal proteins (such as milk proteins, meat proteins and egg proteins); vegetable proteins (such as soy protein, wheat protein, rice protein, and pea protein); mixtures of free amino acids; or combinations thereof. Milk proteins such as casein, whey proteins and soy proteins are particularly preferred. The composition may also contain a source of carbohydrates and a source of fat.

If the nutritional formula includes a fat source, the fat source preferably provides about 5% to about 55% of the energy of the nutritional formula; for example about 20% to about 50% of the energy. The lipids making up the fat source may be any suitable fat or fat mixtures. Vegetable fats are particularly suitable; for example soy oil, palm oil, coconut oil, safflower oil, sunflower oil, corn oil, canola oil, lecithins, and the like. Animal fats such as milk fats may also be added if desired.

A source of carbohydrate may be added to the nutritional formula. It preferably provides about 40% to about 80% of the energy of the nutritional composition. Any suitable carbohydrates may be used, for example sucrose, lactose, glucose, fructose, corn syrup solids, and maltodextrins, and mixtures thereof. Dietary fiber may also be added if desired. If used, it preferably comprises up to about 5% of the energy of the nutritional formula. The dietary fiber may be from any suitable origin, including for example soy, pea, oat, pectin, guar gum, gum arabic, and fructooligosaccharides. Suitable vitamins and minerals may be included in the nutritional formula in an amount to meet the appropriate guidelines.

One or more food grade emulsifiers may be incorporated into the nutritional formula if desired; for example diacetyl tartaric acid esters of mono- and di-glycerides, lecithin and mono- and di-glycerides. Similarly suitable salts and stabilizers may be included.

The nutritional formula intended improving or preventing age-related functional deficits is preferably enterally administrable; for example in the form of a powder, a liquid concentrate, or a ready-to-drink beverage. If it is desired to produce a powdered nutritional formula, the homogenized mixture is transferred to a suitable drying apparatus such as a spray drier or freeze drier and converted to powder.

In another embodiment, a usual food product may be enriched with the combination according to the present invention. For example, a fermented milk, a yogurt, a fresh cheese, a renneted milk, a confectionery bar, breakfast cereal flakes or bars, drinks, milk powders, soy-based products, non-milk fermented products or nutritional supplements for clinical nutrition. Then, the amount of the molecule that stimulates energy metabolism is preferably of at least about 50 ppm by weight and the antioxidant is preferably of at least 10 ppm by weight.

The food composition according to the present invention can prevent or delay mitochondrial dysfunctions occuring during aging by modulating and/or regulating expression of genes linked to energy metabolism of the cell.

Preferably, target genes are those genes involved in (I) energy production: glycolysis, gluconeogenesis, oxidative phosphorylation (respiratory complexes I, II, III, IV, COQ10, ATPsynthase, adenine nucleotide translocase), β-oxidation and tri-carboxylic acid cycle (2) mitochondria biogenesis: membrane components (cardiolipin, PUFAS), protein carriers (ADP/ATP, carnitine, phosphate), proteins synthesis (3) proteases (neutral alkaline protease) (4) ROS production and detoxification (Mn-SOD, Glutathione, UCP) (5) modulators of inflammation.

As target genes the following non exhaustive gene list includes genes involved in:

ATP generation (brain creatine kinase, muscle creatine kinase, mito sarcomeric creatine kinase, ATP synthase, Adenine nucleotide translocase, creatine transporter, tricarboxilate carrier, phosphate transporter, . . . ),

glycolysis (alpha-enolase, glucose-6-phosphate dehydrogenase, glucose-6-phosphatase, pyruvate kinase, phosphoglycerate kinase . . . ),

gluconeogenesis (glucose-6 phosphatase, glucose 1,6-bis phosphatase, . . . ),

β-oxidation (camitine carrier, palmitoyl. Camitine transferase . . . . )

inflammatory response (cox-2, cyclophilin C-AP, lysozyme C . . . ),

mitochondria biogenesis (mitochondria LON protease, HSP70 . . . ),

fatty acid synthesis (fatty acid synthase, stearoyl-CoA desaturase, . . . )

cardiolipin synthesis (PA :CTP cytidylyl transferase . . . ),

protein turnover (proteasome subunit, ribosomal proteins, . . . . ),

stress response (NF-κ-B-p65, I-κ-B α chain . . . ),

thiol protease (cathepsin H and D.),

and other genes (thyroid hormone receptor, glutamine synthase.), for example.

The food composition according to the present invention can also provide multiple benefits by improving age-related functional deficits e.g. in skeletal and cardiac muscle function, vascular function, cognitive function, vision, hearing, olfaction, skin and coat quality, bone and joint health, renal health, gut function, immune function, insulin sensitivity, inflammatory processes, and ultimately increasing longevity in mammals.

According to another aspect, this invention relates to the preparation of a composition intended to prevent or restore age-related functional deficits in mammals. This prearation includes the use of a combination that is able to mimic the effects of caloric restriction on gene expression, which combination comprises at least one molecule that stimulates energy metabolism of the cell and at least one antioxidant. The molecule and antioxidant have been described above.

According to a further aspect of the invention, a method to prevent or restore age-related functional deficits in mammals is provided. This method comprises administering to the mammal a food composition comprising a combination being able to mimic the effects of caloric restriction on gene expression, said combination containing at least one molecule that stimulates energy metabolism of the cell and at least one antioxidant.

The composition may be administered to the mammal as a supplement to the normal diet or as a component of a nutritionally complete food. It is preferred to prepare a nutritionally complete food as described above.

Preferably, the amount of the food composition to be consumed by the mammal to obtain a beneficial effect will depend upon its size, its type, and its age. However an amount of said molecule of at least 1 mg per kg of body weight per day and an amount of the antioxidant of at least 0.025 mg per kg of body weight per day, would usually be adequate.

Administering to a pet or human, a food composition as described above, results in an improved mitochondria function, also mimicking the effects of caloric restriction on gene expression without limiting calorie intake and side effects.

### EXAMPLES

The following examples are given by way of illustration only and in no way should be construed as limiting the subject matter of the present application. All percentages are given by weight unless otherwise indicated.

### Example 1

### Effect of Dietary Interventions With Antioxidants and Activators of Mitochondria Metabolism in a Murine Model by Gene Expression Profiling in Skeletal Muscle

Study Design:

Dietary intervention was of 3 months, all animal groups were fed Ad lib except for the group of caloric restricted mice which as fed 67% of the daily food consumed by the control Ad lib group. Animal weight was measured once a week.

Animals:

Male mice C57/B16 were obtained from Iffa credo (France) at 9 weeks of age. Upon arrival mice were housed by groups of 6 animals. After 3 weeks adaptation, mice (12 weeks old) were randomized in 6 groups (A to E) of 12 mice each and housed individually. Dietary intervention was of 3 months; mice had free access to water and were submitted to 12 hours light and dark cycles.

Diets:

The control diet (diet A) composed of 18% proteins (soy and whey), 11% fat (soybean oil), 59% carbohydrates (starch+sucrose) and 10% cellulose was supplemented with either ginkgo biloba extract (diet E), or a cocktail of antioxidants comprising vitamin C, vitamin E, grape seed extract and cysteine (diet C) and/or L-camitine (diet D and F respectively). For caloric restriction (diet B) fat, starch and sucrose were reduced to provide 67% of the daily calorie consumption of the Ad-lib control group while providing 100% for proteins, minerals and vitamins. These diets are as follows:

Diet A—Control: 18% proteins (soy and whey), 11% fat, 59% carbohydrates, 5% cellulose.

Diet B—Caloric restriction: 18% proteins (soy and whey), 7.7% fat, 32.5% carbohydrates, 5% cellulose

Diet C—Cocktail of antioxidants: Diet A+0.19% vit C, 0.03% vit E, 0.075% grape seed extract, 0.4% cysteine.

Diet D: Diet A+0.3% L-carnitine+cocktail of antioxidants of diet C.

Diet E: Diet A+0.0375% Ginkgo biloba extract (Linnea)

Diet F: Diet A+0.3% L-camitine

RNA Preparation:

Mice were decapitated and dissected rapidly. Skeletal muscles (gastrocnemius) were immersed in RNAlatter (Ambion) and frozen at −80° C. until use. For RNA extraction, muscles were homogenized with ceramic beads (FastPrep, Q-Biogene) and the RNA extracted with Totally RNA kit (Ambion). The quality of the RNA was checked by Agilent technology. RNA pools from four mice each were created and hybridized to Affymetrix Murine U74Av2 high-density oligonucleotide microarrays.

Results

As a first assessment, the five experimental diets were compared to the control diet and clustered (hierarchical clustering) using Spotfire. With this approach, differential gene expression profiles indicate that (1) the two diets containing L-carnitine and caloric restriction belong to the same cluster (2) the diet containing both the antioxidant cocktail and L-carnitine is the most similar to caloric restriction and (3) the antioxidant cocktail & ginkgo form a separate group.

### Example 2

### Dry Pet Food

A feed mixture is made up of about 58% by weight of corn, about 5.5% by weight of corn gluten, about 22% by weight of chicken meal, 2.5% dried chicory, 1% carnitine, and 1% creatine for stimulation of energy metabolism, 0.1% Vit C, vit E (150 IU/kg), 0.05% grape seed proanthocyanidin extract and 1% cysteine as antioxidant, salts, vitamins and minerals making up the remainder.

The fed mixture is fed into a preconditioner and moistened. The moistened feed is then fed into an extruder-cooker and gelatinized. The gelatinized matrix leaving the extruder is forced through a die and extruded. The extrudate is cut into pieces suitable for feeding to dogs, dried at about 110° C. for about 20 minutes, and cooled to form pellets.

This dry dog food is able to improve or restore the age-related deficits in dogs.

### Example 3

### Dry Pet Food

A feed mixture is prepared as in example 1, using 2% carnitine for stimulation of energy metabolism and 0.05% ginkgo biloba extract as antioxidant. Then, the fed mixture is processed as in example 1. The dry dog food is also particularly intended to improve or restore the age-related deficits in dogs.

### Example 4

### Wet Canned Pet Food

A mixture is prepared from 73% of poultry carcass, pig lungs and beef liver (ground), 16% of wheat flour, 2% of dyes, vitamins, and inorganic salts, and 2% of carnitine for stimulation of energy metabolism and 0.4% green tea as antioxidant.

This mixture is emulsified at 12° C. and extruded in the form of a pudding which is then cooked at a temperature of 90° C. It is cooled to 30° C. and cut in chunks. 45% of these chunks are mixed with 55% of a sauce prepared from 98% of water, 1% of dye, and 1% of guar gum. Tinplate cans are filled and sterilized at 125° C. for 40 min.

### Example 5

### Wet Canned Pet Food

A mixture is prepared from 56% of poultry carcass, pig lungs and pig liver (ground), 13% of fish, 16% of wheat flour, 2% of plasma, 10.8% of water, 2.2% of dyes, 1% of semi refined kappa carrageenan, inorganic salts and 9% oil rich in monounsaturated fatty acids (olive oil) and 1% creatine for stimulation of energy metabolism and 1% taurine as antioxidant. This mixture is emulsified at 12° C. and extruded in the form of a pudding which is then cooked at a temperature of 90° C. It is cooled to 30° C. and cut in chunks.

30% of these chunks (having a water content of 58%) is incorporated in a base prepared from 23% of poultry carcass, 1% of guar gum, 1% of dye and aroma and 75% of water. Tinplate cans are then filled and sterilized at 127° C. for 60 min.

### Example 6

### Nutritional Formula

A nutritional composition is prepared, and which contains for 100 g of powder 15% of protein hydrolysate, 25% of fats, 55% carbohydrates (including 37% maltodextrin, 6% starch, and 12% sucrose), traces of vitamins and oligoelements to meet daily requirements, 2% minerals and 3% moisture and 2% pyruvate for stimulation of energy metabolism and 1% carnosine or carnosine precursor as antioxidant.

13 g of this powder is mixed in 100 ml of water. The obtained formula is particularly intended for reversing age-related gene expression alterations and restore or prevent age-related functional deficits in humans.

What is claimed is:

1. A food composition intended to prevent or restore age-related functional deficits in mammals, which comprises an edible substance and a combination that is able to mimic the effects of caloric restriction on gene expression, the combination containing (a) at least one molecule that stimulates energy metabolism of the cell in an amount effective to cause such stimulation, and (b) at least one antioxidant in an amount effective to reduce or prevent oxidative damage resulting from disruption of ATP/ADP or NAD+/NADH homeostasis due to increased substrate availability or utilization in aged mitochondria.

2. The food composition according to claim 1, wherein the molecule stimulates energy metabolism of mitochondria.

3. The food composition according to claim 1, wherein the molecule is L-carnitine, creatine, a monounsaturated or polyunsaturated fatty acid, cardiolipin, nicotinamide, or a carbohydrate or natural source containing such a molecule.

4. The food composition according to claim 1, wherein the amount of the molecule is of at least 1 mg to 1 g per kg of body weight per day.

5. The food composition according to claim 1, wherein the antioxidant is a source of a thiol or a compound that upregulates their biosynthesis in vivo.

6. The food composition according to claim 5, wherein the antioxidant is lipoic acid, cysteine, cystine, methionine, S-adenosyl-methionine, taurine, glutathione or a natural source thereof.

7. The food composition according to claim 1, wherein the amount of the antioxidant is of at least 0.025 mg to 250 mg per kg of body weight per day.

8. The food composition according to claim 1, in which the antioxidant is used in combination with a further antioxidant.

9. The food composition according to claim 8, in which the further antioxidant is vitamin C, vitamin E, carotenoids, ubiquinones, tea catechins, coffee extracts containing polyphenols and/or diterpenes, ginkgo biloba extracts, grape or grape seed extracts rich in proanthocyanidins, spice extracts, soy extracts containing isoflavones, a related phytoestrogen or other source of flavonoids having antioxidant activity, or a compound that upregulates a cell antioxidant defense.

10. The food composition according to claim 8, in which the further antioxidant is ursodeoxycholic acid, ursolic acid, ginseng, a gingenoside, or a natural source thereof.

11. The food composition according to claim 1, which further comprises an effective amount of a prebiotic micro-organism, a probiotic micro-organism, or both.

12. A pet food or dietary supplement comprising the food composition according to claim 1.

13. A nutritionally complete human food composition or a dietary supplement comprising the food composition according to claim 1.

14. A method for preventing or delaying mitochondria dysfunction occurring in a mammal during aging, which method comprises administering to a mammal in need of such treatment a combination that is able to mimic the effects of caloric restriction on gene expression, the combination containing (a) at least one molecule that stimulates energy metabolism of the cell in an amount effective to cause such stimulation, and (b) at least one antioxidant in an amount effective to reduce or prevent oxidative damage resulting from disruption of ATP/ADP or NAD+/NADH homeostasis due to increased substrate availability or utilization in aged mitochondria, and being administered in an amount effective to modulate or regulate expression of genes linked to energy metaboism.

15. The method of claim 14, wherein the combination is administered to the mammal by way of a food composition that is consumed by the mammal.

16. The method of claim 15, wherein the food composition contains further additives to improve one or more of skeletal and cardiac muscle function, vascular function, cognitive function, vision, hearing, olfaction, skin and coat quality, bone and joint health, renal health, gut function, immune function, insulin sensitivity, or inflammatory processes.

17. A method for preventing or restoring age-related functional deficits in mammals which comprises administering to a mammal in need of such treatment a combination that is able to mimic the effects of caloric restriction on gene expression, the combination containing (a) at least one molecule that stimulates energy metabolism of the cell in an amount effective to cause such stimulation, and (b) at least one antioxidant in an amount effective to reduce or prevent oxidative damage resulting from disruption of ATP/ADP or NAD+/NADH homeostasis due to increased substrate availability or utilization in aged mitochondria.

18. The method of claim 17, wherein the molecule stimulates energy metabolism of mitochondria.

19. The method of claim 17, wherein the combination is administered in an amount effective to modulate or regulate expression of genes linked to energy metabolism.

20. The method of claim 17, wherein the molecule that stimulates energy metabolism of the cell is L-camitine, creatine, fatty acids (mono and polyunsaturated, particularly omega-3 fatty acids), cardiolipin, nicotinamide or a carbohydrate or natural source containing such a molecule.

21. The method of claim 17, wherein the antioxidant is a source of thiols (e.g. Lipoic acid, cysteine, cystine, methionine, S-adenosyl-methionine, taurine, glutathione and natural sources thereof), or a compound that upregulates their biosynthesis in vivo.

22. The method of claim 17, in which the antioxidant is used in association with a further antioxidant.

23. The method of claim 22 wherein the further antioxidant is vitamin C, vitamin E, carotenoids, ubiquinones, tea catechins, coffee extracts containing polyphenols and/or diterpenes, ginkgo biloba extracts, grape or grape seed extracts rich in proanthocyanidins, spice extracts, soy extracts containing isoflavones and related phytoestrogens and other sources of flavonoids with antioxidant activity or compounds that upregulate cell antioxidant defense.

24. The method of claim 17, wherein the amount of the molecule is at least 1 mg to 1 g per kg of body weight of the mammal per day and the amount of the antioxidant is at least 0.025 mg to 250 mg per kg of body weight of the mammal per day.

25. A method to prevent or restore age-related functional deficits in mammals, comprising administering to the mammal the food composition according to claim 1.
